(19) United States
(12) Patent Application Publication
(10) Pub. No.: US 2007/0231803 A1
(43) Pub. Date: Oct. 4, 2007

(54) MULTIPLEX PCR MIXTURES AND KITS CONTAINING THE SAME

(76) Inventor: Mark A. Jensen, West Chester, PA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/394,588

(22) Filed: Mar. 31, 2006

(51) Int. Cl. C12Q 1/68; C12P 19/34

(52) U.S. Cl. 435/6; 435/91.2

(57) ABSTRACT

A multiplex PCR reaction mixture is provided. In one embodiment, the reaction mixture contains a plurality of primer pairs that bind to genomic DNA for producing predetermined amplification products of a range of different sizes, where each primer pair is at a concentration that is selected for production of a pre-determined amount of amplification product if the genomic DNA is intact. Also provided are methods of using the reaction mixture.

### BACKGROUND

In general terms, the quality of the results obtained from a genomic assay (e.g., the degree of correspondence between the actual copy number of a genomic locus and the prediction made about the copy number of that genomic locus) is often dependent on the quality of the genomic DNA sample used to perform the assay. Since the quality of a genomic DNA sample employed in a genomic assay may vary greatly, the quality of results obtained from a genomic assay may also vary greatly. For example, in certain cases, the genomic DNA in a sample employed in a genomic assay may be partially or completely degraded, which may make that genomic DNA difficult to effectively amplify and/or label.

### SUMMARY

A multiplex PCR reaction mixture is provided. In one embodiment, the reaction mixture contains a plurality of primer pairs that bind to genomic DNA for producing predetermined amplification products of a range of different sizes, where each primer pair is at a concentration that is selected for production of a pre-determined amount of amplification product if the genomic DNA is intact. Also provided are methods of using the reaction mixture.

### BRIEF DESCRIPTION OF THE DRAWINGS

FIG. 1 shows an electropherogram output for a mass-balanced multiplex PCR amplification of a 50 ng female human genomic DNA sample. Analysis was done using a DNA 1000 LabChip on an Agilent 2100 Bioanalyzer.

FIG. 2 shows an electropherogram output for a molar concentration-balanced multiplex PCR amplification of a 40 ng male human genomic DNA sample.

FIG. 3 shows an electropherogram of sonicated human genomic DNA sample.

FIG. 4 shows an electropherogram of mass-balanced multiplex PCR amplification products from a sonicated human genomic DNA sample.

FIG. 5 is a graph showing a comparison of concentrations of amplifiable targets and resulting PCR products. PCR target concentration was normalized relative to a human genomic DNA control sample. The solid line represents the percentage of the total population of sonicated hgDNA with a size greater than the corresponding X-axis value. The six solid dots represent the normalized concentrations of the six multiplex PCR products relative to an amplified control hgDNA sample.

FIG. 6 shows an electropherogram of DNA extracted from formalin fixed paraffin embedded (FFPE) sample. The upper molecular weight marker corresponds to a DNA fragment size of 10380 bp.

FIG. 7 shows an electropherogram of mass balanced PCR of the FFPE sample used to obtain the results shown in FIG. 6.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “mRNA” means messenger RNA.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides.

An “oligonucleotide” generally refers to a nucleotide multimer of about 2 to about 200 nucleotides in length (e.g., about 10 to about 100 nucleotides or about 30 to about 80 nucleotides) while a “polynucleotide” or “nucleic acid” includes a nucleotide multimer having any number of nucleotides. Oligonucleotides may be synthetic

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm² or even less than 10 cm². For example, features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in representative embodiments, known. An array feature is generally homogenous in composition and concentration and the features may be separated by intervening spaces (although arrays without such separation can be fabricated).

The term “substrate” as used herein refers to a surface upon which probes, e.g., an array, may be adhered. Substrates may be porous or non-porous, planar or non-planar over all or a portion of their surface. Glass slides are the most common substrate for arrays, although fused silica, silicon, plastic and other materials are also suitable. A substrate may contain more than one array.

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. The support can be planar, nonplanar or a combination thereof. The support can be porous or non-porous. In certain embodiments, the collections of oligonucleotide elements employed herein are present on a surface of the same planar support, e.g., in the form of an array. It should be understood that the terms “probe” and “target” are relative terms and that a molecule considered as a probe in certain assays may function as a target in other assays.

“Addressable sets of probes” and analogous terms refer to the multiple known regions of different moieties of known characteristics (e.g., base sequence composition) supported by or intended to be supported by an array surface, such that each location is associated with a moiety of a known characteristic and such that properties of a target moiety can be determined based on the location on the array surface to which the target moiety binds under stringent conditions.

In certain embodiments, an array is contacted with a nucleic acid sample under stringent assay conditions, i.e., conditions that are compatible with producing bound pairs of biopolymers of sufficient affinity to provide for the desired level of specificity in the assay while being less compatible to the formation of binding pairs between binding members of insufficient affinity. Stringent assay conditions are the summation or combination (totality) of both binding conditions and wash conditions for removing unbound molecules from the array.

As known in the art, “stringent hybridization conditions” and “stringent hybridization wash conditions” in the context of nucleic acid hybridization are sequence dependent, and are different under different experimental parameters. Stringent hybridization conditions include, but are not limited to, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization in 0.5 M NaHPO4, 7% sodium dodecyl sulfate (SDS), 1 mM EDTA at 65° C., and washing in 0.1×SSC/0.1% SDS at 68° C. can be performed. Additional stringent hybridization conditions include hybridization at 60° C. or higher and 3×SSC (450 mM sodium chloride/45 mM sodium citrate) or incubation at 42° C. in a solution containing 30% formamide, IM NaCl, 0.5% sodium sarcosine, 50 mM MES, pH 6.5. Those of ordinary skill will readily recognize that alternative but comparable hybridization and wash conditions can be utilized to provide conditions of similar stringency.

Wash conditions used to remove unbound nucleic acids may include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Other methods of agitation can be used, e.g., shaking, spinning, and the like.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate. The term “highly stringent hybridization conditions” as used herein refers to conditions that are compatible to produce complexes between complementary binding members, i.e., between immobilized probes and complementary sample nucleic acids, but which does not result in any substantial complex formation between non-complementary nucleic acids (e.g., any complex formation which cannot be detected by normalizing against background signals to interfeature areas and/or control regions on the array).

Additional hybridization methods are described in references describing CGH techniques (Kallioniemi et al., Science 1992; 258:818-821 and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol. 1981; 21:470-480 and Angerer et al., In Genetic Engineering: Principles and Methods, Setlow and Hollaender, Eds. Vol 7, pgs 43-65 (Plenum Press, New York 1985). See also U.S. Pat. Nos. 6,335,167; 6,197,501; 5,830,645; and 5,665,549; the disclosures of which are herein incorporated by reference.

The term “sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiment, samples are a complex mixture of molecules, e.g., comprising about 50 or more different molecules, about 100 or more different molecules, about 200 or more different molecules, about 500 or more different molecules, about 1000 or more different molecules, about 5000 or more different molecules, about 10,000 or more molecules, etc.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell or cell type. Genomic sequences include, but are not limited to, those involved in the maintenance, replication, segregation, and generation of higher order structures (e.g. folding and compaction of DNA in chromatin and chromosomes), or other functions, if any, of nucleic acids, as well as all the coding regions and their corresponding regulatory elements needed to produce and maintain each virus, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

As used herein, a “test nucleic acid sample” or “test nucleic acids” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

Similarly, “reference genomic acids” or a “reference genomic sample” refers to genomic nucleic acids comprising sequences whose quantity or degree of representation (e.g., copy number) or sequence identity is to be compared with a test nucleic acids. A “reference nucleic acid sample” may be derived independently from a “test nucleic acid sample,” i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked in the sample.

If a surface-bound polynucleotide or primer “corresponds to” a chromosome, the polynucleotide usually contains a sequence of nucleic acids that is unique to that chromosome. Accordingly, a surface-bound polynucleotide that corresponds to a particular chromosome usually specifically hybridizes to a labeled nucleic acid made from that chromosome, relative to labeled nucleic acids made from other chromosomes. Array features, because they usually contain surface-bound polynucleotides, can also correspond to a chromosome.

“Hybridizing”, “annealing” and “binding”, with respect to nucleic acids, are used interchangeably. If a polynucleotide “binds to”, “corresponds to” or is “for” a certain RNA or DNA, the polynucleotide base pairs with, i.e., specifically hybridizes to, that RNA or DNA under stringent conditions, e.g., the conditions employed in a PCR reaction. As will be discussed in greater detail below, a particular RNA or DNA and a polynucleotide for that particular RNA or DNA, or complement thereof, usually contain at least one region of contiguous nucleotides that is identical in sequence.

A “primer” can be extended from its 3′ end by the action of a polymerase. An oligonucleotides that cannot be extended from it 3′ end by the action of a polymerase is not a primer.

A “CGH array” or “aCGH array” refers to an array that can be used to compare DNA samples for relative differences in copy number. In general, an aCGH array can be used in any assay in which it is desirable to scan a genome with a sample of nucleic acids. For example, an aCGH array can be used in location analysis as described in U.S. Pat. No. 6,410,243, the entirety of which is incorporated herein and thus can also be referred to as a “location analysis array” or an “array for ChIP-chip analysis.” In certain aspects, a CGH array provides probes for screening or scanning a genome of an organism and comprises probes from a plurality of regions of the genome.

In one aspect, the array comprises probe sequences for scanning an entire chromosome arm, wherein probes targets are separated by about 500 bp or more, about 1 kb or more, about 5 kb or more, about 10 kb or more, about 25 kb or more, about 50 kb or more, about 100 kb or more, about 250 kb or more, about 500 kb or more and about 1 Mb or more. In another aspect, the array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

In certain aspects, in constructing arrays, both coding and non-coding genomic regions are included as probes, whereby “coding region” refers to a region comprising one or more exons that is transcribed into an mRNA product and from there translated into a protein product, while by non-coding region is meant any sequences outside of the exon regions, where such regions may include regulatory sequences, e.g., promoters, enhancers, untranslated but transcribed regions, introns, origins of replication, telomeres, etc. In certain embodiments, one can have at least some of the probes directed to non-coding regions and others directed to coding regions. In certain embodiments, one can have all of the probes directed to non-coding sequences and such sequences can, optionally, be all non-transcribed sequences (e.g., intergenic regions including regulatory sequences such as promoters and/or enhancers lying outside of transcribed regions).

In certain aspects, an array may be optimized for one type of genome scanning application compared to another, for example, the array can be enriched for intergenic regions compared to coding regions for a location analysis application.

In some embodiments, at least 5% of the polynucleotide probes on the solid support hybridize to regulatory regions of a nucleotide sample of interest while other embodiments may have at least 30% of the polynucleotide probes on the solid support hybridize to exonic regions of a nucleotide sample of interest. In yet other embodiments, at least 50% of the polynucleotide probes on the solid support hybridize to intergenic regions (e.g., non-coding regions which exclude introns and untranslated regions, i.e, comprise non-transcribed sequences) of a nucleotide sample of interest.

In certain aspects, probes on the array represent random selection of genomic sequences (e.g., both coding and noncoding). However, in other aspects, particular regions of the genome are selected for representation on the array, e.g., such as CpG islands, genes belonging to particular pathways of interest or whose expression and/or copy number are associated with particular physiological responses of interest (e.g., disease, such a cancer, drug resistance, toxological responses and the like). In certain aspects, where particular genes are identified as being of interest, intergenic regions proximal to those genes are included on the array along with, optionally, all or portions of the coding sequence corresponding to the genes. In one aspect, at least about 100 bp, 500 bp, 1,000 bp, 5,000 bp, 10,000 kb or even 100,000 kb of genomic DNA upstream of a transcriptional start site is represented on the array in discrete or overlapping sequence probes. In certain aspects, at least one probe sequence comprises a motif sequence to which a protein of interest (e.g., such as a transcription factor) is known or suspected to bind.

In certain aspects, repetitive sequences are excluded as probes on the arrays. However, in another aspect, repetitive sequences are included.

The choice of nucleic acids to use as probes may be influenced by prior knowledge of the association of a particular chromosome or chromosomal region with certain disease conditions. International Application WO 93/18186 provides a list of exemplary chromosomal abnormalities and associated diseases, which are described in the scientific literature. Alternatively, whole genome screening to identify new regions subject to frequent changes in copy number can be performed using the methods of the present invention discussed further below.

In some embodiments, previously identified regions from a particular chromosomal region of interest are used as probes. In certain embodiments, the array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

In certain aspects, the array includes probes to sequences associated with diseases associated with chromosomal imbalances for prenatal testing. For example, in one aspect, the array comprises probes complementary to all or a portion of chromosome 21 (e.g., Down's syndrome), all or a portion of the X chromosome (e.g., to detect an X chromosome deficiency as in Turner's Syndrome) and/or all or a portion of the Y chromosome Klinefelter Syndrome (to detect duplication of an X chromosome and the presence of a Y chromosome), all or a portion of chromosome 7 (e.g., to detect William's Syndrome), all or a portion of chromosome 8 (e.g., to detect Langer-Giedon Syndrome), all or a portion of chromosome 15 (e.g., to detect Prader-Willi or Angelman's Syndrome, all or a portion of chromosome 22 (e.g., to detect Di George's syndrome).

Other “themed” arrays may be fabricated, for example, arrays including whose duplications or deletions are associated with specific types of cancer (e.g., breast cancer, prostate cancer and the like). The selection of such arrays may be based on patient information such as familial inheritance of particular genetic abnormalities. In certain aspects, an array for scanning an entire genome is first contacted with a sample and then a higher-resolution array is selected based on the results of such scanning. Themed arrays also can be fabricated for use in gene expression assays, for example, to detect expression of genes involved in selected pathways of interest, or genes associated with particular diseases of interest.

In one embodiment, a plurality of probes on the array are selected to have a duplex Tₘ within a predetermined range. For example, in one aspect, about 50% or more of the probes have a duplex Tₘ within a temperature range of about 75° C. to about 85° C. In one embodiment, at least 80% of the polynucleotide probes have a duplex Tₘ within a temperature range of about 75° C. to about 85° C., within a range of about 77° C. to about 83° C., within a range of from about 78° C. to about 82° C. or within a range from about 79° C. to about 82° C. In one aspect, about 50% or more of the probes on an array have range of Tₘ's of less than about 4° C., less than about 3° C., or even less than about 2° C., e.g., less than about 1.5° C., less than about 1.0° C. or about 0.5° C.

In certain embodiments, the probes on the microarray have a nucleotide length in the range of at least 30 nucleotides to 200 nucleotides, or in the range of about 30 to about 150 nucleotides. In other embodiments, about 50% or more of the polynucleotide probes on the solid support have the same nucleotide length, and that length may be about 60 nucleotides.

In still other aspects, probes on the array comprise at least coding sequences.

In one aspect, probes represent sequences from an organism such as Drosophila melanogaster, Caenorhabditis elegans, yeast, zebrafish, a mouse, a rat, a domestic animal, a companion animal, a primate, a human, etc. In certain aspects, probes representing sequences from different organisms are provided on a single substrate, e.g., on a plurality of different arrays.

A “CGH assay” using an aCGH array can be generally performed as follows. In one embodiment, a population of nucleic acids contacted with an aCGH array comprises at least two sets of nucleic acid populations, which can be derived from different sample sources. For example, in one aspect, a target population contacted with the array comprises a set of target molecules from a reference sample and from a test sample. In one aspect, the reference sample is from an organism having a known genotype and/or phenotype, while the test sample has an unknown genotype and/or phenotype or a genotype and/or phenotype that is known and is different from that of the reference sample. For example, in one aspect, the reference sample is from a healthy patient while the test sample is from a patient suspected of having cancer or known to have cancer.

In one embodiment, a target population being contacted to an array in a given assay comprises at least two sets of target populations that are differentially labeled (e.g., by spectrally distinguishable labels). In one aspect, control target molecules in a target population are also provided as two sets, e.g., a first set labeled with a first label and a second set labeled with a second label corresponding to first and second labels being used to label reference and test target molecules, respectively.

In one aspect, the control target molecules in a population are present at a level comparable to a haploid amount of a gene represented in the target population. In another aspect, the control target molecules are present at a level comparable to a diploid amount of a gene. In still another aspect, the control target molecules are present at a level that is different from a haploid or diploid amount of a gene represented in the target population. The relative proportions of complexes formed labeled with the first label vs. the second label can be used to evaluate relative copy numbers of targets found in the two samples.

In certain aspects, test and reference populations of nucleic acids may be applied separately to separate but identical arrays (e.g., having identical probe molecules) and the signals from each array can be compared to determine relative copy numbers of the nucleic acids in the test and reference populations.

Methods to fabricate arrays are described in detail in U.S. Pat. Nos. 6,242,266; 6,232,072; 6,180,351; 6,171,797 and 6,323,043. As already mentioned, these references are incorporated herein by reference. Drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample and then read. Reading of an array may be accomplished by illuminating the array and reading the location and intensity of resulting fluorescence at multiple regions on each feature of the array. For example, a scanner may be used for this purpose is the AGILENT MICROARRAY SCANNER manufactured by Agilent Technologies, Palo, Alto, Calif. or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. Scanning typically produces a scanned image of the array which may be directly inputted to a feature extraction system for direct processing and/or saved in a computer storage device for subsequent processing. However, arrays may be read by any other methods or apparatus than the foregoing, other reading methods including other optical techniques or electrical techniques (where each feature is provided with an electrode to detect bonding at that feature in a manner disclosed in U.S. Pat. Nos. 6,251,685, 6,221,583 and elsewhere).

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

By “remote location” is meant a location other than the location at which an array is present and hybridization occurs. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart.

“Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of surface-bound polynucleotides, as is commonly known in the art and described below, is not a mixture of surface-bound polynucleotides because the species of surface-bound polynucleotides are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

The term “absolute concentration” refers to the concentration of a compound in a liquid, where the concentration is expressed as the weight of the compound per volume of liquid, e.g., ng/μl. If two or more compounds are at the same absolute concentration in a liquid, the same weight of the compound is present in the same unit volume of each of the liquids, regardless of the molecular weight of the compound. In certain embodiments, a PCR amplification reaction that produces a plurality of products that are all at the same absolute concentration is referred to as a “mass-balanced” multiplex PCR amplification reaction.

The term “molar concentration” refers to the concentration of a compound in a liquid, where the concentration is expressed as the number of molecules (as expressed in Moles, for example) of the compound per volume of liquid, e.g., nM or nmol/μl. If two or more compounds are at the same molar concentration in a liquid, the same number of molecules of the compound are present in the same unit volume of each of the liquids. In certain embodiments, a PCR amplification reaction that produces a plurality of products that are all at the same molar concentration is referred to as a “molar concentration-balanced” multiplex PCR amplification reaction.

Unless otherwise indicated, where an “amount” of a compound is expressed, that amount may be an absolute concentration or a molar concentration. For example, if the same amount of a plurality of amplification products are present in a PCR reaction, the amplification products may have the same absolute concentration or the same molar concentration.

A “yield-balanced” multiplex PCR amplification may be either a “mass-balanced” reaction or a “molar concentration-balanced” reaction, as discussed above.

A “size ladder” is what is provided if a plurality (i.e., two or more) of PCR amplification products are resolved by size using a separation device, e.g., in a capillary device or in a gel, e.g., an agarose or acrylamide gel.

### DETAILED DESCRIPTION

A multiplex PCR reaction mixture is provided. In one embodiment, the reaction mixture contains a plurality of primer pairs that bind to genomic DNA for producing predetermined amplification products of a range of different sizes, where each primer pair is at a concentration that is selected for production of a pre-determined amount of amplification product if the genomic DNA is intact.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Representative embodiments of the subject methods are described in greater detail below, followed by a description of representative protocols in which the subject methods find use. Finally, kits for performing the subject method are described.

Multiplex PCR Reaction Mixture

A multiplex polymerase chain reaction (PCR) reaction mixture is provided. In certain aspects, the subject multiplex PCR reaction mixture contains a plurality of primer pairs as well a reaction buffer (which be pH buffered and may include salt, e.g., MgCl₂ and other components necessary for PCR), nucleotides, e.g., dGTP, dATP, dTTP and dCTP and a DNA polymerase, e.g., a thermostable DNA polymerase. In certain embodiments, the reaction mixture may further contain a sample.

Exemplary reaction buffers and DNA polymerases that may be employed in the subject reaction mixture include those described in (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.) and, as such, are not discussed in any detail. Reaction buffers and DNA polymerases suitable for PCR may be purchased from a variety of suppliers, e.g., Invitrogen (Carlsbad, Calif.), Qiagen (Valencia, Calif.) and Stratagene (La Jolla, Calif.). Exemplary polymerases include Taq, Pfu, Pwo, UITma and Vent, although many other polymerases may be employed in certain embodiments. Guidance for the reaction components suitable for use with a polymerase as well as suitable conditions for its use, is found in the literature supplied with the polymerase.

As noted above, the subject reaction mixture contains a plurality of primer pairs (e.g., two or more, e.g., 3 or more, 4 or more, 5 or more, 6 or more, 7 or more, 8 or more, 9 or more, or 10 or more primer pairs) that bind to genomic DNA for producing predetermined amplification products of a range of different lengths.

When employed in PCR, each primer pair of the mixture is expected to produce an amplification product of known length that may be within the range of 50 bp to 3 kb, 60 bp to 2 kb, 80 bp to 1.5 kb, 90 bp to 1.2 kb, although primer pairs that produce amplification products outside of this length range may be employed in certain embodiments. Collectively, the plurality of primers in the reaction mixture produce a corresponding plurality of amplification products. In certain embodiments, the amplification products are physically resolvable by size such that the amount of each amplification product can be determined. For example, if there are five amplification products, those amplification products may be resolved and the amount of each of the amplification products may be determined. In certain embodiments, the amplification products may have different lengths, and produce a size ladder when separated by length such that they are distributed across a size range. In certain embodiments, the size of the amplification products may be distributed across a size range that is between 50 bp to 3 kb in size, although in certain embodiments a wider or narrower range may be employed. In one embodiment, the primer pairs produce a size ladder of amplification products that are distributed between 80 bp and 600 bp in length, 100 bp and 1 kb in length, or 100 bp and 2 kb in length. Depending on the range of length of the amplification products and the number of primer pairs employed, the length difference between any two amplification products may be at least 50 bp, at last 100 bp or at least 200 bp, for example. In one embodiment, the plurality of primer pairs may produce a ladder of amplification products, where the size difference between consecutive amplification products is about 50 to 150 bp, e.g., about 80 to 120 bp, or 150 to 250 bp, e.g., 180 to 200 bp, in length.

In one embodiment, the primer pairs bind to and amplify products from different regions of the genome under analysis. Each primer pair of the plurality of primer pairs may amplify a product from a different chromosome of the genome under analysis. In certain embodiments, the primer pairs should bind to and amplify a single copy locus of the genome under analysis, i.e., a unique sequence that is represented once per haploid genome.

As would be apparent from the preceding description, the nucleotide sequences of the primers used in the subject reaction mixtures may vary greatly. However, since the genomes of many eukaryotic organisms have been sequenced and those sequences have been annotated and deposited into public databases such as NCBI's Genbank Database, the primers that could be used in the instant methods are readily designed. In certain embodiments, detectably labeled (e.g., fluorescent) primers may be employed.

The primers of the reaction mixture may be designed to have similar thermodynamic properties, e.g., similar Tms, G/C content, hairpin stability, and in certain embodiments may all be of a similar length, e.g., from 18 to 30 nt, e.g., 20 to 25 nt in length.

As noted above, each primer pair is at a concentration that is selected for production of a pre-determined amount of amplification product if the genomic DNA to which those primer pairs bind is intact. A pre-determined amount of amplification product may be any selected amount of amplification product, where the selected amount of amplification product is measurable. Unlike conventional multiplex PCR reaction mixtures, the instant PCR reaction mixture does not contain the same amount of each of the primers. Each primer pair of an instant reaction mixture is present at a different concentration (molar or by w/v) to the other primer pairs.

In one embodiment, the primer pairs of a subject reaction mixture are each at a concentration that provides for production of a plurality of amplification products that are each at the same amount (i.e., the same molar concentration or the same absolute concentration) if an intact DNA genome is present in the reaction. As such, the pre-determined amount of amplification product may be an amount relative to the amount of another amplification product.

In certain embodiments, if the primer pairs are employed at a concentrations that provides for production of a plurality of amplification products that are each at the same amount (i.e., the same molar concentration or the same absolute concentration) if an intact DNA genome is present in the reaction, the amount of each of the amplification products may be within +/−20%, +/−10% or +/−5% of the average amount (by weight or moles) of amplification product.

Since, in general terms, small products are amplified more efficiently than larger products and not all primer pairs have the same efficiency, a PCR reaction mixture that contains the same amount of primers, or an arbitrarily chosen amount of primers, does not produce a pre-determined amount of amplification product. The primer pairs of the instant reaction mixture, in general, are titrated in the absence and presence of the other primers using an intact genome to identify a concentration that provides for a particular amount of amplification product. As such, the amount of each primer pair used is validated prior to its use. In such titration assays, the primers are tested at different concentrations and under different conditions (e.g., by varying the temperatures, incubation times and ramping speeds) against genomic DNA that is not cross-linked and intact, i.e., substantially undegraded (e.g., containing genomic DNA that is less than about 10% degraded, where degradation of genomic DNA may be calculated by determining the amount of the genomic DNA that is below about 100 kb in length, relative to the amount of genomic DNA that is above about 100 kb in length) to identify and select optimal primer concentrations and PCR conditions.

The amount of primer present in a subject reaction mixture may vary greatly. In certain embodiments, each primer pair may be present at an amount in the range of 1 μM to 100 μM, e.g., 3 μM to 50 μM. In a 50 μl reaction these amounts would correspond to concentrations of 0.02 μM to 2 μM, e.g. 0.06 μM to 1 μM. Likewise, the amount of amplification product produced may also vary greatly. In certain embodiments the amount of each amplification product is at least detectable on the instrument used for detection, and may be in the range of 1 pg/μl to 10 pg/μ10 pg/μl to 100 pg/μl, 100 pg/μl to 1 ng/μl, 1 ng/μl to 10 ng/μl or 10 ng/μl to 100 ng/μl, for example. In certain embodiments that employ primers that provide the same absolute amount of amplification products, the reaction mix may contain more molecules of the primer pairs for the shorter products then for the longer products.

In certain embodiments, a subject reaction mix may further contain a genomic sample. The genomic sample present in the subject reaction mix may contain genomic DNA or an amplified version thereof (e.g., genomic DNA amplified using the methods of Lage et al, Genome Res. 2003 13: 294-307 or published patent application US20040241658, for example) from the nuclei of eukaryotic cells. In exemplary embodiments, the genomic sample may contain genomic DNA from a mammalian cell such a human, mouse, rat or monkey cell.

The cells used to produce a genomic sample may be cultured cells or cells of a clinical sample, e.g., a tissue biopsy, scrape or lavage and, in certain embodiments, may or may not be cells of a forensic sample (i.e., cells of a sample collected at a crime scene). In particular embodiments, the genomic sample may be derived (e.g., made from) from an archived sample (which may or may not be a cellular sample) that has been stored prior to use (e.g., stored prior to labeling or stored prior to extraction of genomic DNA from the sample). If employed, an archived sample may have been stored under any condition, e.g., at below room temperature (e.g., frozen such as at about −80° C., at about −20° C. or at about 4° C.), at room temperature (e.g., at about 20° C.), above room temperature, at below atmospheric pressure (e.g., in a vacuum), above atmospheric pressure (e.g., under pressure) or at atmospheric pressure (about 760 Torr) for several hours, days, weeks or years prior to use, for example. In particular embodiments, the genomic sample may contain DNA that may be cross-linked, e.g., by chemical treatment by a cross-linker such as formalin or formaldehyde, and may, in certain embodiments, be obtained from cross-linked formalin fixed paraffin embedded (FFPE) sample.

The genomic DNA content of a genomic sample may be undetermined (i.e., known or unknown), prior to performing the subject methods. Likewise, the integrity of the genomic DNA of a genomic sample may be undetermined prior to performing the subject methods. In particular embodiments, the genomic DNA of a genomic sample may be intact, i.e., substantially undegraded (e.g., containing genomic DNA that is less than about 10% degraded). In other embodiments, the genomic DNA of a genomic sample may be substantially degraded (i.e., containing genomic DNA that is at least about 10% degraded, e.g., at least about 50%, at least about 80%, at least about 90% or at least about 95% or about 99% degraded), where degradation of genomic DNA may be calculated by determining the amount of the genomic DNA that is below about 100 kb in length, relative to the amount of genomic DNA that is above about 100 kb in length. Although there is no requirement to know the amount of genomic DNA that is present in a genomic sample, genomic DNA at concentrations of about 0.1 pg/μl to about 1 pg/μl, about 1 pg/μl to about 10 pg/μl, 10 pg/μl to about 0.1 ng/μl, 0.1 ng/μl to about 1 ng/μl, about 1 ng/μl to about 10 ng/μl, about 10 ng/μl to about 100 ng/μl, about 100 ng/μl to about 1 μg/μl of genomic DNA are readily employed.

A genomic sample is obtained by, for example, receiving a genomic sample or producing a genomic sample from cells. Methods for making such genomic samples are generally well known in the art and described in the publications discussed in the background section herein, and in well known laboratory manuals (e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y. for example).

Method of Sample Analysis

A method assessing a genomic sample is also provided. In general terms, this method includes: a) making the above-described multiplex PCR reaction mixture; b) maintaining the multiplex PCR reaction mixture under conditions suitable for PCR; and c) assessing the amplification products produced by the PCR. In certain embodiments, the abundance of each amplification product is assessed to provide an assessment of the quality (e.g., integrity) of the sample.

As will be discussed in greater detail below, the abundance of the molecular weight amplification products provides an evaluation of the integrity of the genomic DNA of a genomic sample. In certain embodiments, for example, the PCR reaction may yield a set of amplification products in which the abundance of the higher molecular weight products is lower than the pre-determined amounts expected for those products, i.e., lower than would be expected if the genomic sample contained intact genomic DNA. In this case, the genomic sample may contain degraded or cross-linked genomic DNA, rather than intact DNA.

In certain embodiments, results obtained from a subject assay may be compared to control results to provide an evaluation of the genomic sample. The control results may be obtained using a control genomic sample, e.g., a genomic sample containing a genome of known integrity.

PCR conditions of interest include those well known in the art (e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y. for example). The amounts of the amplification products may be assessed after any number of rounds of PCR amplification (i.e., successive cycles of denaturation, re-naturation and polymerization). In certain embodiments, the amounts of amplification products may be assessed a stage at which the nucleic acid amplification occurs linearly (i.e., during the linear phase of the amplification reaction) or after the reaction rate has reached a plateau. In certain embodiments the amounts of amplification products may be assessed after 12 and before 30 successive rounds of amplification, e.g., 12 to 16 rounds, 16 to 20 rounds, 20 to 24 rounds or 24 to 30 rounds of amplification. In general, the number of rounds of application employed provides an amount of amplification product that is detectable using the detection system employed. The optimal number of rounds of amplification employed in the subject methods may vary primer set to primer set, as discussed above. The optimal number of rounds of amplification for each genomic sample is readily determinable. In certain embodiments, the amount of the primers employed in the PCR reactions is limiting.

After amplification, the amount of the amplification products may be assessed. The amount of amplification products may be assessed by any suitable means, including, but not limited to: separating the products according to their size using a separation device (for example, a column, gel or filter) and independently measuring the amount of each of the separated products by, e.g., a) contacting the separated products with a detectable (e.g., fluorescent) DNA binding agent and assessing the amount of bound agent, b) by detecting absorbance at 260 nm, or, c) detecting the presence of a detectable label if a detectably labeled primer was employed in the amplification reaction. The methods described above are readily automated. In certain embodiments, a microfluidic system may be employed for analysis of amplification products. One representative system that may be employed is a microcapillary device such as the DNA 7500 LabChip and Bioanalyzer of Agilent Technologies (Palo Alto, Calif.). In one embodiment, the amplification products may be labeled and hybridized to a polynucleotide array containing surface bound polynucleotides that bind to those products. The level of binding of the labeled amplification products to the array indicates the amount of the amplification products in the sample.

The amounts of the amplification products may be assessed to provide a qualitative or quantitative evaluation of the genomic sample. For example, the amounts of each of the amplification products may be determined, and compared to the amounts that would be expected if the genomic sample contains intact genomic DNA. If the amounts of each of the amplification products are the same as would be expected if the genomic sample contained intact genomic DNA, then the genomic sample likely contains intact genomic DNA. In another example, the amounts of each of the larger molecular weight amplification products may be lower than expected, indicating the genomic DNA in the sample is degraded.

As demonstrated in the examples section below, the results obtained from these assays may be graphed, and, in certain embodiments, the average fragment size of a genomic sample may be calculated. In another embodiments, a line of best fit may be drawn, and the degree of deviance of the line from the line that would be expected if the genomic DNA is intact indicates the degree of degradation of the sample.

In one embodiment, the results are compared to results obtained from a control genomic sample that, in certain embodiments, may contain genomic DNA of pre-determined (i.e., known) integrity, e.g., substantially undegraded (e.g., containing genomic DNA that is less than about 10% degraded) or substantially degraded. In certain embodiments the control genomic sample contains genomic DNA of a quality that is known to be suitable for use in an array-based genome analysis (e.g., CGH) assay.

In certain embodiments, the control sample may be made from the same species, tissue type and/or cell-type as the test sample. As would be apparent to one of skill in the art, amplification reactions for test and control sample, if employed, may be performed in parallel or in series. Results obtained using a test sample may be compared to results obtained using a first control sample and a second control sample, where the first control sample may contain substantially undegraded genomic DNA and the second control sample may contain substantially degraded DNA.

In general terms, the closer the amounts of the amplification products are to the amounts expected if the genomic sample contains intact genomic DNA, the more intact the genomic DNA of the test sample.

The above-described protocols may be employed in a variety of methods, including in: a) methods of identifying a test genomic sample suitable for use in an array-based genome hybridization assay, b) methods of identifying a test genomic sample suitable for amplification, c) methods of identifying samples that amplified uniformly, d) methods of selecting a test genomic sample for use and e) methods of selecting a method for amplifying a test genomic sample. In general terms, these methods include analyzing the amplification products of the above-described PCR reaction to produce results, and, on the basis of those results, indicating whether a test sample is of a suitable quality for further use. Accordingly, the methods described above have a particular utility as a quality control step in providing samples of sufficient quality for use in, for example, array-based genome experiments, amplification protocols, or other genome analysis methods, e.g., SNP detection and DNA fingerprinting.

Methods of identifying a test genomic sample suitable for use in an array-based comparative genome hybridization assay generally include: a) performing the instant methods on the test genomic sample to produce an assessment of the integrity of the test genomic sample and b) determining whether the assessment is above a threshold. In general, a test genomic sample having an assessment above a threshold indicates that the test genomic sample is suitable for use in an array-based genome hybridization assay, or, in other methods, suitable for amplification.

Since many amplification methods (e.g., those described in Lage et al, Genome Res. 2003 13: 294-307 or published patent application US20040241658) require a relatively intact genome template for efficient amplification to occur, the instant methods may be readily employed to determine if a genomic sample is suitable for amplification. As would be readily apparent, if a genomic sample is deemed to have an integrity that is below a threshold integrity, that genomic sample may not be suitable for amplification. Likewise, if a genomic sample is deemed to have an integrity that is above a threshold integrity, the genomic sample may be suitable for amplification. In these methods, the integrity of a genomic sample may be tested using the above methods and, on the basis of the results obtained, the genomic sample may be deemed suitable or unsuitable for amplification. If the genomic sample is deemed suitable for amplification, it may be labeled and employed in a genomic assay (e.g., a CGH assay) described in greater detail below. The instant methods may also be employed to determine the size of DNA fragments that may be amplified for subsequent analysis by such means as Short tandem repeats (STR) or mitochondrial DNA sequencing.

Methods of selecting a test genomic sample generally include: a) performing the instant methods on a plurality (e.g., 2 or more, e.g., 5 or more, 10 or more, 50 or more or 100 or more) of test genomic samples to produce a numerical assessment for each test sample; and b) selecting one or more test genomic samples from the plurality of test genomic samples based on whether the numerical assessment for each sample is above the threshold.

In certain embodiments of these methods, particularly if the amplification products are separated by size or other physical means, the degree of smearing or laddering of the amplification products may also be taken into consideration in deciding whether a test sample is of a suitable quality for further use.

Kits

Kits for use in accordance with the subject methods are also provided. The kits at least, as described above, a plurality of primer pairs that bind to genomic DNA for producing predetermined amplification products of a range of different sizes, where each primer pair is at a concentration that is selected for production of a pre-determined amount of amplification product if the genomic DNA is intact.

A kit may include one or more of: a control genomic sample that contains a genome that is substantially undegraded or substantially degraded, reagents for labeling a genomic sample, a polymerase, e.g., a thermostable polymerase, or reaction buffer components for performing PCR, e.g., MgCl₂ and nucleotides, etc.

A subject kit may further include one or more additional components necessary for carrying out an array-based genome assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay. Such kits also typically include instructions for use in practicing array-based assays.

The kits may also include a computer readable medium including and instructions that may include directions for use of the invention.

The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

Utility

Samples evaluated, or, in certain embodiments, selected according to the above methods may be employed in a genome analysis assay that may be array based. Such samples may be employed in, for example, a fingerprinting assay, a SNP detection assay, sequence detection assay, or a CGH assay that can be employed to evaluate CpG islant methylation, location, or copy number, for example. In one embodiment, such assays may be employed for the quantitative comparison of copy number of one nucleic acid sequence in a first collection of nucleic acid molecules relative to the copy number of the same sequence in a second collection.

The arrays employed in CGH assays contain polynucleotides immobilized on a solid support. Array platforms for performing the array-based methods are generally well known in the art (e.g., see Pinkel et al., Nat. Genet. (1998) 20:207-211; Hodgson et al., Nat. Genet. (2001) 29:459-464; Wilhelm et al., Cancer Res. (2002) 62: 957-960) and, as such, need not be described herein in any great detail. In general, CGH arrays contain a plurality (i.e., at least about 100, at least about 500, at least about 1000, at least about 2000, at least about 5000, at least about 10,000, at least about 20,000, usually up to about 100,000 or more) of addressable features that are linked to a planar solid support. Features on a subject array usually contain a polynucleotide that hybridizes with, i.e., binds to, genomic sequences from a cell. Accordingly, such “comparative genome hybridization arrays”, for short “CGH arrays” typically have a plurality of different BACs, cDNAs, oligonucleotides, or inserts from phage or plasmids, etc., that are addressably arrayed. As such, CGH arrays usually contain surface bound polynucleotides that are about 10-200 bases in length, about 201-5000 bases in length, about 5001-50,000 bases in length, or about 50,001-200,000 bases in length, depending on the platform used.

In particular embodiments, CGH arrays containing surface-bound oligonucleotides, i.e., oligonucleotides of 10 to 100 nucleotides and up to 200 nucleotides in length, find particular use in the subject methods.

In general, the subject assays involve labeling a test and a reference genomic sample to make two labeled populations of nucleic acids which may be distinguishably labeled, contacting the labeled populations of nucleic acids with an array of surface bound polynucleotides under specific hybridization conditions, and analyzing any data obtained from hybridization of the nucleic acids to the surface bound polynucleotides. Such methods are generally well known in the art (see, e.g., Pinkel et al., Nat. Genet. (1998) 20:207-211; Hodgson et al., Nat. Genet. (2001) 29:459-464; Wilhelm et al., Cancer Res. (2002) 62: 957-960)) and, as such, need not be described herein in any great detail.

Two different genomic samples may be differentially labeled, where the different genomic samples may include an “experimental” sample, i.e., a sample of interest, and a “control” sample to which the experimental sample may be compared. In certain embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell type may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells, or in different phases of the cell cycle) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells.

The genomic sample (containing intact, fragmented or enzymatically amplified chromosomes, or amplified fragments of the same), are distinguishably labeled using methods that are well known in the art (e.g., primer, extension, random-priming, nick translation, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). The samples are usually labeled using “distinguishable” labels in that the labels that can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), fluorescein and Texas red (Dupont, Bostan Mass.) and POPRO3 TOPRO3 (Molecular Probes, Eugene, Oreg.). Further suitable distinguishable detectable labels may be found in Kricka et al. (Ann Clin Biochem. 39:114-29, 2002).

The labeling reactions produce a first and second population of labeled nucleic acids that correspond to the test and reference chromosome compositions, respectively. After nucleic acid purification and any optional pre-hybridization steps to suppress repetitive sequences (e.g., hybridization with Cot-1 DNA), the populations of labeled nucleic acids are contacted to an array of surface bound polynucleotides, as discussed above, under conditions such that nucleic acid hybridization to the surface bound polynucleotides can occur, e.g., in a buffer containing 50% formamide, 5×SSC and 1% SDS at 42° C., or in a buffer containing 5×SSC and 1% SDS at 65° C., both with a wash of 0.2×SSC and 0.1% SDS at 65° C.

The labeled nucleic acids can be contacted to the surface bound polynucleotides serially, or, in other embodiments, simultaneously (i.e., the labeled nucleic acids are mixed prior to their contacting with the surface-bound polynucleotides). Depending on how the nucleic acid populations are labeled (e.g., if they are distinguishably or indistinguishably labeled), the populations may be contacted with the same array or different arrays. Where the populations are contacted with different arrays, the different arrays are substantially, if not completely, identical to each other in terms of target feature content and organization.

Standard hybridization techniques (using high stringency hybridization conditions) are used to probe a target nucleic acid array. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985). See also U.S. Pat. Nos. 6,335,167; 6,197,501; 5,830,645; and 5,665,549; the disclosures of which are herein incorporate by reference.

Generally, comparative genome hybridization methods comprise the following major steps: (1) immobilization of polynucleotides on a solid support; (2) pre-hybridization treatment to increase accessibility of support-bound polynucleotides and to reduce nonspecific binding; (3) hybridization of a mixture of labeled nucleic acids to the surface-bound nucleic acids, typically under high stringency conditions; (4) post-hybridization washes to remove nucleic acid fragments not bound to the solid support polynucleotides; and (5) detection of the hybridized labeled nucleic acids. The reagents used in each of these steps and their conditions for use vary depending on the particular application.

As indicated above, hybridization is carried out under suitable hybridization conditions, which may vary in stringency as desired. In certain embodiments, highly stringent hybridization conditions may be employed. The term “high stringent hybridization conditions” as used herein refers to conditions that are compatible to produce nucleic acid binding complexes on an array surface between complementary binding members, i.e., between the surface-bound polynucleotides and complementary labeled nucleic acids in a sample. Representative high stringency assay conditions that may be employed in these embodiments are provided above.

The above hybridization step may include agitation of the immobilized polynucleotides and the sample of labeled nucleic acids, where the agitation may be accomplished using any convenient protocol, e.g., shaking, rotating, spinning, and the like.

Following hybridization, the array-surface bound polynucleotides are typically washed to remove unbound labeled nucleic acids. Washing may be performed using any convenient washing protocol, where the washing conditions are typically stringent, as described above.

Following hybridization and washing, as described above, the hybridization of the labeled nucleic acids to the targets is then detected using standard techniques so that the surface of immobilized targets, e.g., the array, is read. Reading of the resultant hybridized array may be accomplished by illuminating the array and reading the location and intensity of resulting fluorescence at each feature of the array to detect any binding complexes on the surface of the array. For example, a scanner may be used for this purpose, which is similar to the AGILENT MICROARRAY SCANNER available from Agilent Technologies, Palo Alto, Calif. Other suitable devices and methods are described in U.S. patent application Ser. No. 09/846,125 “Reading Multi-Featured Arrays” by Dorsel et al.; and U.S. Pat. No. 6,406,849, which references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of nucleic acids, and are suitable for some embodiments.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been present in the sample, or whether or not a pattern indicates a particular condition of an organism from which the sample came).

In certain embodiments, the subject methods include a step of transmitting data or results from at least one of the detecting and deriving steps, also referred to herein as evaluating, as described above, to a remote location. By “remote location” is meant a location other than the location at which the array is present and hybridization occurs. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart.

“Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

Accordingly, a pair of chromosome compositions is labeled to make two populations of labeled nucleic acids, the nucleic acids contacted with an array of surface-bound polynucleotides, and the level of labeled nucleic acids bound to each surface-bound polynucleotide is assessed.

In certain embodiments, a surface-bound polynucleotide is assessed by determining the level of binding of the population of labeled nucleic acids to that polynucleotide. The term “level of binding” means any assessment of binding (e.g. a quantitative or qualitative, relative or absolute assessment) usually done, as is known in the art, by detecting signal (i.e., pixel brightness) from the label associated with the labeled nucleic acids. Since the level of binding of labeled nucleic acid to a surface-bound polynucleotide is proportional to the level of bound label, the level of binding of labeled nucleic acid is usually determined by assessing the amount of label associated with the surface-bound polynucleotide.

In certain embodiments, a surface-bound polynucleotide may be assessed by evaluating its binding to two populations of nucleic acids that are distinguishably labeled. In these embodiments, for a single surface-bound polynucleotide of interest, the results obtained from hybridization with a first population of labeled nucleic acids may be compared to results obtained from hybridization with the second population of nucleic acids, usually after normalization of the data. The results may be expressed using any convenient means, e.g., as a number or numerical ratio, etc.

By “normalization” is meant that data corresponding to the two populations of nucleic acids are globally normalized to each other, and/or normalized to data obtained from controls (e.g., internal controls produce data that are predicted to equal in value in all of the data groups). Normalization generally involves multiplying each numerical value for one data group by a value that allows the direct comparison of those amounts to amounts in a second data group. Several normalization strategies have been described (Quackenbush et al, Nat Genet. 32 Suppl:496-501, 2002, Bilban et al Curr Issues Mol Biol. 4:57-64, 2002, Finkelstein et al, Plant Mol Biol. 48(1-2): 119-31, 2002, and Hegde et al, Biotechniques. 29:548-554, 2000). Specific examples of normalization suitable for use in the subject methods include linear normalization methods, non-linear normalization methods, e.g., using lowess local regression to paired data as a function of signal intensity, signal-dependent non-linear normalization, qspline normalization and spatial normalization, as described in Workman et al., (Genome Biol. 2002 3, 1-16). In certain embodiments, the numerical value associated with a feature signal is converted into a log number, either before or after normalization occurs. Data may be normalized to data obtained using the data obtained from a support-bound polynucleotide for a chromosome of known concentration in any of the chromosome compositions.

Accordingly, binding of a surface-bound polynucleotide to a labeled population of nucleic acids may be assessed. In most embodiments, the assessment provides a numerical assessment of binding, and that numeral may correspond to an absolute level of binding, a relative level of binding, or a qualitative (e.g., presence or absence) or a quantitative level of binding. Accordingly, a binding assessment may be expressed as a ratio, whole number, or any fraction thereof.

CGH assays may be used to identify abnormal nucleic acid copy number and mapping or investigating of chromosomal abnormalities associated with disease, e.g., cancer for example.

### EXAMPLE 1

### Yield-Balanced Multiplex PCR

An exemplary yield-balanced multiplex PCR amplification contains six unique target human genome sequences. The PCR products range in size from 108 to 491 bps. Each target is located on a different chromosome and contains a 60-base probe sequence that is present on the CGH arrays. The target priming site chromosome, gene name and transcript ID for the probe sequence are shown in the following table:

| Gene Name | Chr | Transcript ID | Fragment Size |
|---|---|---|---|
| INHBA | 7 | NM_002192 | 108 bp |
| BRE | 2 | NM_004899 | 166 bp |
| NUP | 6 | NM_005124 | 238 bp |
| B4GALT2 | 1 | NM_003490 | 309 bp |
| SYN3 | 22 | NM_003490 | 411 bp |
| CDK5RAP2 | 9 | NM_018249 | 491 bp |

The sequence of the primers used are shown in the following table:

| | |
|---|---|
| Primer name | Sequence |

| | |
|---|---|
| INHBA 508-21 | AGTCAACAGTTTTCAGATTG |
| | (SEQ ID NO: 1) |
| INHBA 599-20 | GGCCAGTAAAGTATGTGCAG |
| | (SEQ ID NO: 2) |
| BRE 428 | CTCTAGGCCCACTGCTAT |
| | (SEQ ID NO: 3) |
| BRE 574 | TAAGTGCAACAAGTTGTAGG |
| | (SEQ ID NO: 4) |
| NUP153 591 | TCCGAAACCACTGTCAAT |
| | (SEQ ID NO: 5) |
| NUP153 810 | TGTCACCCAGAGATACTGC |
| | (SEQ ID NO: 6) |
| B4GALT 423 | ACCTAGTTGCTGTTGCCTAA |
| | (SEQ ID NO: 7) |
| B4GALT 715 | GGCAGGGCTCTAAGTCAG |
| | (SEQ ID NO: 8) |
| SYN3 572 | CAGGCCTTGTAATTGTAGCA |
| | (SEQ ID NO: 9) |
| SYN3 970 | GGCCCTGAACTGTACC |
| | (SEQ ID NO: 10) |
| CDKRAP2 157 | ACTCTTGGGCAACTCAAAGC |
| | (SEQ ID NO: 11) |
| CDKRAP2 628 | CTCTCATGCGCTCTCTGATT |
| | (SEQ ID NO: 12) |

Primer pairs are shown below:

1d INHBA 508-21/INHBA 599-20
IIb BRE 428/BRE 574
IVa NUP153 591/NUP153 810
V B4GALT 423/B4GALT 715
VI SYN3 572/SYN3 970
VIIa CDKRAP2 157/CDKRAP2 628

The amount of oligonucleotide primer used in a PCR reaction is chosen to produce equal yields of all amplification products. The product yield can be balanced in, for example, two ways, either by mass (i.e., by the weight of the molecules produced) or by molar concentration (i.e., by the number of molecules produced). Balanced yields may be obtained using primer pairs at concentrations that provide balanced yields, and by optimizing the PCR conditions used, e.g., optimizing the synthesis time in thermal cycling. Conventional multiplex PCR (mPCR) typically uses equal amounts of primers. Equal amounts of primers are not used in yield-balanced multiplex PCR methods.

### EXAMPLE 2

### Mass-Balanced Multiplex PCR

In this multiplex PCR (mPCR) approach, PCR primer concentrations are adjusted to produce equal masses (i.e., equal weights of molecules) of each amplicon. Since the products all have different molecular weights, each amplicon is produced at a different molar concentration (as measured in, e.g., nmoles/vol).

When run on a gel and stained, the products of mass-balanced multiplex PCR (mPCR) amplification produces equal band intensities because the intensity of a band on a gel is determined by how much amplicon mass is available to the intercalating dye. Mass-balanced multiplex PCR may be employed in conjunction with, for example, gel detection, because gels have a relatively narrow dynamic range over which product can be visualized and measured.

The molar concentration of DNA in the sample that is a larger size than the amplicon target, determines how much amplicon of that size is generated in the mPCR. In order to determine the size molar concentration profile of the sample, the sample mPCR output may be compared with the mPCR output for a non-degraded sample, where all the available PCR targets have the same molar concentration. The mean DNA fragment size in the sample is greater than the largest PCR amplicon. The size molar concentration distribution in the unknown sample may be determined from this comparison.

An exemplary protocol for performing mass-balanced PCR is set forth below.

1. Prepare the following mix per reaction:
5 μl 10× Qiagen Taq buffer
2 μl 25 mM MgCl₂
5 μl 2.5 mM dNTPs

2. Add the following primer stocks to the buffer mix:
20 pM each primer of primer set Id;
16.9 pM each primer of primer set IIb;
8.5 pM each primer of primer set IVa;
16.9 pM each primer of primer set V;
7.1 pM each primer of primer set VI;
7.1 pM each primer of primer set VIIa;

3. Add IU Qiagen HotStartTaq

4. Add H₂O to a final volume of 49 μl and add 1.0 μl DNA sample (hgDNA)

5. Run the following cycling conditions:

| | |
|---|---|
| 15 mins at 95° C. then: | 15 s at 95° C. |
| | 60 s at 57° C. |
| | 60 s at 72° C. |
| | repeat 28 cycles |
| | 10 min. at 72° C. |

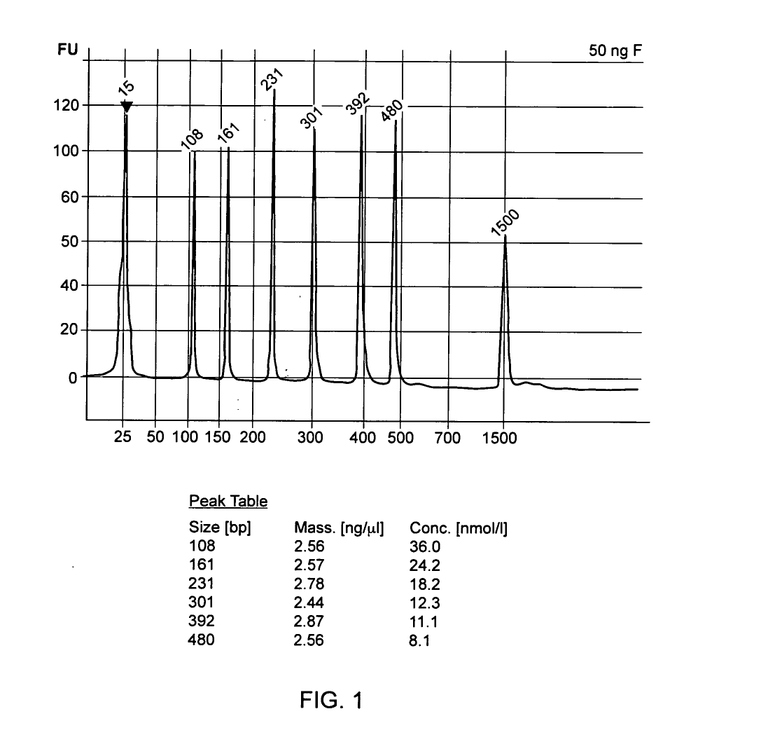

FIG. 1 illustrates results obtained using this protocol.

### EXAMPLE 3

### Molar Concentration Balanced Multiplex PCR

Another approach to mPCR is to balance the molar concentration (in nmole/vol.) of each amplicon that is generated by the mPCR. This is again accomplished by adjusting the primer concentrations, such that in a non-degraded sample, all the amplicons are produced at equal molar concentrations, number of molecules. This approach can be used in conjunction with a capillary detection device such as a Bioanalyzer. The Bioanalyzer has 3-log dynamic range. The molar concentrations of mPCR products can be measured over a significant dynamic range.

Since the mPCR reaction is balanced to generate all the amplicons at equal molar concentrations from a non-degraded sample, the yield of the smallest amplicon can serve as an internal standard for comparison with the larger amplicons. A relative decrease in the molar concentration yield in the larger amplicons can be directly attributed to a decrease in concentration (copy number) of available DNA targets of the corresponding size. The size vs. mPCR product molar concentration yield relationship can be directly translated into a size vs. available target concentration in the sample. From this relationship, a mean-amplifiable DNA size can be determined. This in turn, provides a direct indication of the degree to which the sample DNA is degraded or chemically compromised, as in the case of formaldehyde crosslinking of formalin fixed paraffin embedded (FFPE) tissue.

An exemplary protocol for performing molar concentration-balanced PCR is set forth below.

1. Prepare the following mix per reaction:
5 μl 10× Qiagen Taq buffer
2 μl 25 mM MgCl₂
5 μl 12.5 mM dNTPs

2. Add the following primer stocks to the buffer mix:
5.5 pM each primer of primer set Id;
8.5 pM each primer of primer set IIb;
6.25 pM each primer of primer set IVa;
27.5 pM each primer of primer set V;
16.8 pM each primer of primer set VI;
24 pM each primer of primer set VIIa;

3. Add 2.5 U Qiagen HotStartTaq

4. Add H₂O to a final volume of 49 μl and add 1.0 μl DNA sample (hgDNA)

5. Run the following cycling conditions:

| | |
|---|---|
| 15 mins at 95° C. then: | 15 s at 95° C. |
| | 60 s at 57° C. |
| | 120 s at 72° C. |
| | repeat 28 cycles |
| | 10 min. at 72° C. |

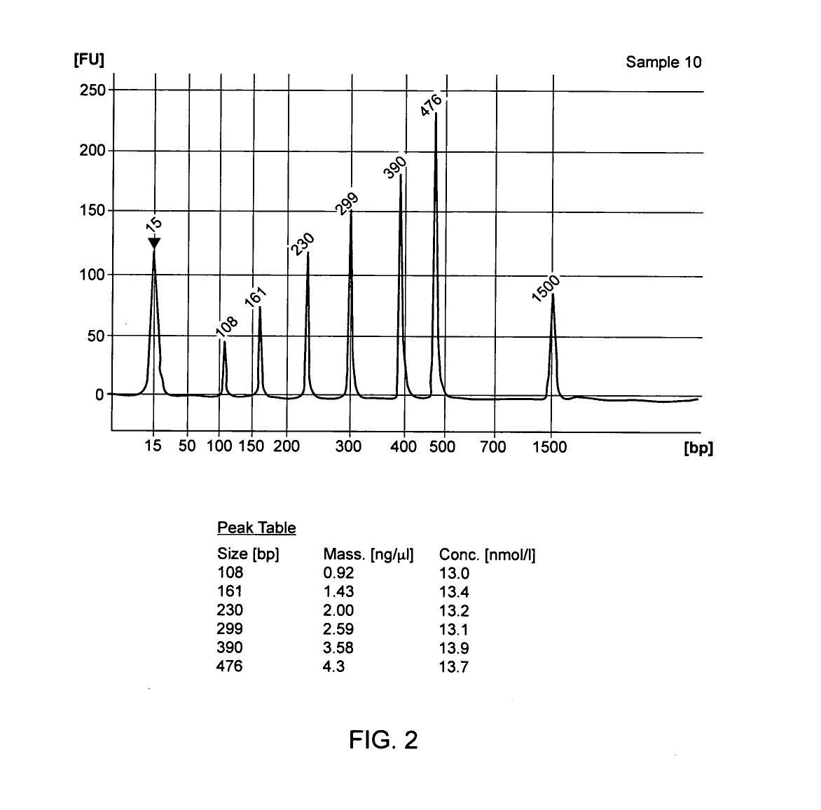

FIG. 2 illustrates results obtained using this protocol.

### EXAMPLE 4

### Yield Balanced Multiplex PCR of Sonicated Genomic DNA

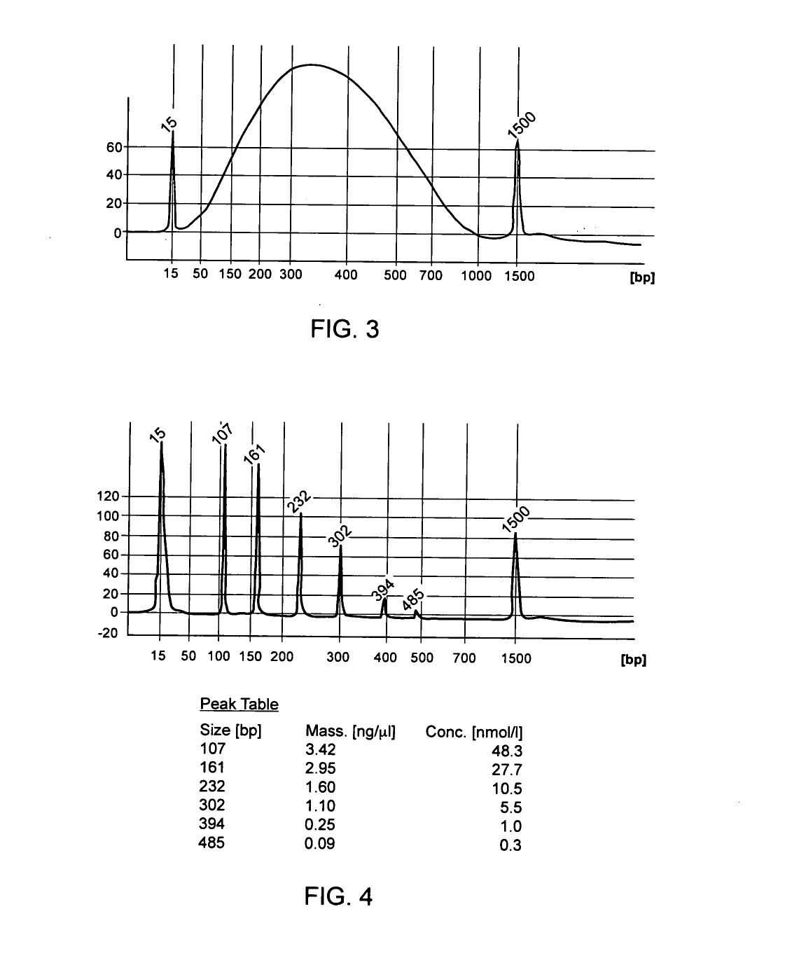

To demonstrate the effectiveness of yield-balanced multiplex PCR analysis, a sonicated human DNA sample was characterized by electrophoresis and mass-balanced multiplex PCR. The output for the electrophoresis and the PCR are shown in FIGS. 3 and 4, respectively.

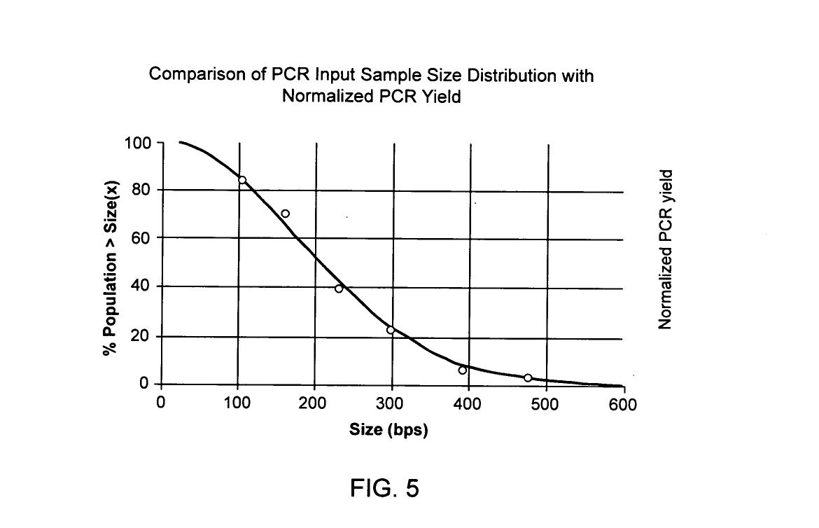

Analysis of the electropherogram and the PCR product yields demonstrate that the relative yield of each of the six PCR products gave an accurate indicated of the concentration of DNA in the sample that was larger than the corresponding PCR amplicon (FIG. 5).

### EXAMPLE 5

### Yield Balanced Multiplex PCR of Crosslinked Genomic DNA

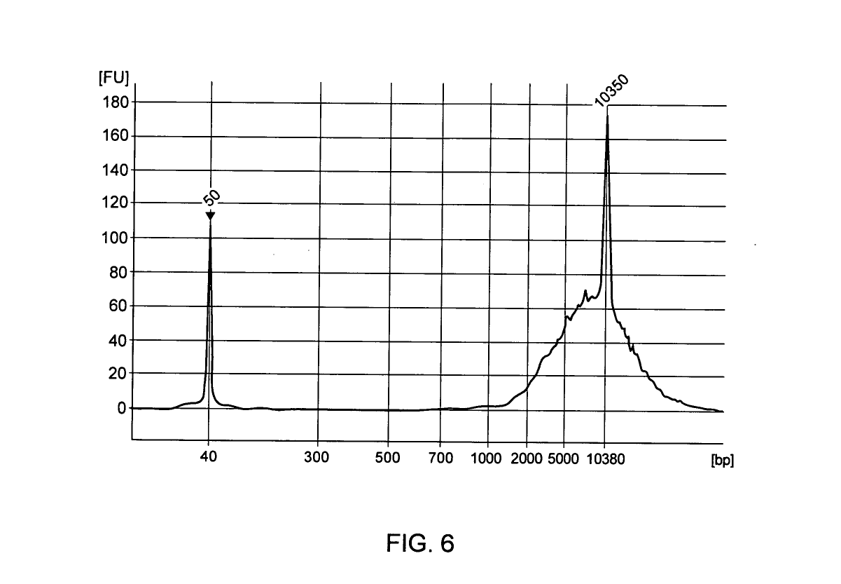

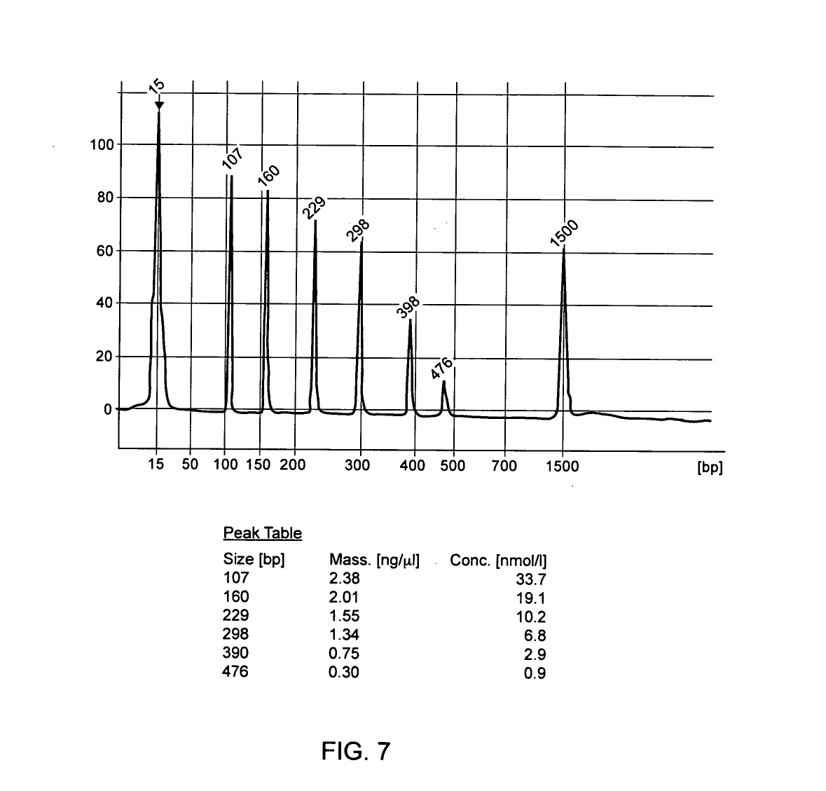

In FFPE samples the isolated DNA often contains a significant level of residual crosslinking. Although a gel or capillary electrophoresis analysis of the sample may indicate mobility consistent with the size of 10 kb (FIG. 6), the mean length of accessible and amplifiable DNA was considerably less. FIGS. 6 and 7, respectively, show an electropherogram of DNA isolated from an FFPE sample and the corresponding multiplex PCR analysis. The decreased amplification yields in the larger PCR products indicate that the length of DNA accessible for amplification is considerably less than the 10 kb size of the unamplified target.

The preceding merely illustrates principles of exemplary embodiments. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A multiplex polymerase chain reaction (PCR) reaction mixture comprising:
a) two or more primer pairs that bind to genomic DNA for producing predetermined amplification products of a range of different sizes, where each primer pair is at a concentration that is selected for production of a predetermined amount of amplification product if said genomic DNA is intact;
b) a polymerase;
c) nucleotides; and
d) reaction buffer.

2. The multiplex PCR reaction mixture of claim 1, wherein each of said primer pairs is at a concentration that provides for production of a plurality of amplification products that are each at the same molar concentration.

3. The multiplex PCR reaction mixture of claim 1, wherein each of said primer pairs is at a concentration that provides for production of a plurality of amplification products that are each at the same absolute concentration.

4. The multiplex PCR reaction mixture of claim 1, wherein said multiplex PCR reaction mixture comprises at least two and less than 10 primer pairs.

5. The multiplex PCR reaction mixture of claim 1, wherein said predetermined amplification products are distributed across a size range that is within the range of 50 bp to about 2 kb.

6. The multiplex PCR reaction mixture of claim 1, wherein said genomic DNA comprises a nuclear genome of a mammalian cell.

7. The multiplex PCR reaction mixture of claim 6, wherein said mammalian cell is a human cell.

8. The multiplex PCR reaction mixture of claim 1, further including a sample.

9. The multiplex PCR reaction mixture of claim 1, wherein said sample is a stored sample.

10. The multiplex PCR reaction mixture of claim 1, wherein said sample comprises genomic DNA of unknown integrity.

11. The multiplex PCR reaction mixture of claim 1, wherein said polymerase is a thermostable DNA polymerase.

12. A method assessing a genomic sample, comprising:
a) making a multiplex PCR reaction mixture of claim 1;
b) combining said multiplex PCR reaction mixture with sample;
c) maintaining said multiplex PCR reaction mixture under PCR conditions; and
d) evaluating said amplification products to assess genomic sample.

13. The method of claim 12, wherein said method comprises size separating said amplification products.

14. The method of claim 12, wherein said evaluating produces results, and said results are compared to control results.

15. The method of claim 14, wherein said control results are obtained from a sample known to contain an intact genome.

16. A method of assessing the integrity of a test genomic sample, comprising:
performing the method of claim 12 on said test genomic sample to produce results; and
comparing said results to reference results;
to produce an assessment of the integrity of said test genomic sample.

17. A method of identifying a test genomic sample suitable for use, comprising:
performing the method of claim 12 on said test genomic sample to produce an assessment of the integrity of said test genomic sample; and
determining whether said assessment is above a threshold;
wherein an assessment above said threshold indicates that said test genomic sample is suitable for use.

18. The method of claim 17, wherein said threshold is arbitrarily selected.

19. The method of claim 17, wherein an assessment above said threshold indicates that said genomic sample is suitable for use in an array-based genome hybridization assay.

20. The method of claim 17, wherein an assessment above said threshold indicates that said genomic sample is suitable for amplification.

21. The method of claim 17, wherein said method indicates the size of DNA fragments in said test genomic sample.

22. A method of selecting a test genomic sample, comprising:
performing the method of claim 12 on a plurality of test genomic samples; and
selecting a test genomic sample from said plurality of test genomic samples based on whether said assessment is above said threshold.

23. A method comprising:
identifying a test genomic sample suitable for use in an array-based comparative genome hybridization assay using the method of claim 22; and
employing said test genomic sample in an array-based genome hybridization assay.

24. The method of claim 23, wherein said employing step comprises:
labeling said test genomic sample to produce a labeled sample;
contacting said labeled sample with an polynucleotide array; and
detecting the presence of binding complexes on the surface of said array to assay said sample.

25. A kit comprising:
two or more primer pairs that bind to genomic DNA for producing predetermined amplification products of a range of different sizes, where each primer pair is at a concentration that is selected for production of a pre-determined amount of amplification product if said genomic DNA is intact.

26. The kit of claim 25, further comprising a control genomic sample having an intact genomic.

27. The kit of claim 25, further comprising a polymerase.

28. The kit of claim 25, further comprising a reaction buffer.

29. The kit of claim 25, further comprising nucleotides.


SEQUENCE LISTING (12 sequences)

SEQ ID NO: 1 - DNA, 20 bp, H. sapiens
agtcaacagttttcagattg

SEQ ID NO: 2 - DNA, 20 bp, H. sapiens
ggccagtaaagtatgtgcag

SEQ ID NO: 3 - DNA, 18 bp, H. sapiens
ctctaggcccactgctat

SEQ ID NO: 4 - DNA, 20 bp, H. sapiens
taagtgcaacaagttgtagg

SEQ ID NO: 5 - DNA, 18 bp, H. sapiens
tccgaaaccactgtcaat

SEQ ID NO: 6 - DNA, 19 bp, H. sapiens
tgtcacccagagatactgc

SEQ ID NO: 7 - DNA, 20 bp, H. sapiens
acctagttgctgttgcctaa

SEQ ID NO: 8 - DNA, 18 bp, H. sapiens
ggcagggctctaagtcag

SEQ ID NO: 9 - DNA, 20 bp, H. sapiens
caggccttgtaattgtagca

SEQ ID NO: 10 - DNA, 16 bp, H. sapiens
ggccctgaactgtacc

SEQ ID NO: 11 - DNA, 20 bp, H. sapiens
actcttgggcaactcaaagc

SEQ ID NO: 12 - DNA, 20 bp, H. sapiens
ctctcatgcgctctctgatt